(19) United States
(12) Patent Application Publication
(10) Pub. No.: US 2014/0056760 A1
(43) Pub. Date: Feb. 27, 2014

(54) APPARATUS AND METHODS FOR DISINFECTING A SURFACE

(71) Applicants: Allan Kenneth Frazer Grugeon Hunt, Brough (GB); John George Chewins, Andover (GB)

(72) Inventors: Allan Kenneth Frazer Grugeon Hunt, Brough (GB); John George Chewins, Andover (GB)

(73) Assignee: BIOQUELL UK LIMITED, Andover, Hampshire (GB)

(21) Appl. No.: 14/112,522

(22) PCT Filed: May 14, 2012
(86) PCT No.: PCT/GB2012/051068

§ 371 Date: Oct. 17, 2013

(30) Foreign Application Priority Data
May 18, 2011 (GB) 1108375.5

(51) Int. Cl. A61L 2/20

(52) U.S. Cl. CPC A61L 2/202
USPC 422/29; 422/186.12

(57) ABSTRACT

The invention relates to improvements in surface disinfection apparatus and methods and in particular to apparatus and methods which may be used to disinfect wounds using high concentration aqueous ozone. The method comprises the steps of flushing an ion exchange means with fluid from a fluid supply and directing the flushing fluid to waste; de-ionising fluid from the fluid supply by passing it through the ion exchange means and directing the process fluid to storage means; and purging the ion exchange means with gas to remove residual process fluid after the de-ionisation process has been completed.

The invention relates to improvements in surface disinfection apparatus and methods and in particular to apparatus and methods which may be used to disinfect wounds using high concentration aqueous ozone.

One system is described in patent specification WO-A-2004/103452 which details an apparatus and method for carrying out the treatment comprising three main components: an apparatus for generating a concentrated aqueous solution of ozone; an apparatus for spraying the ozone solution onto the surface of a limb to be treated; and an apparatus for supporting a limb to be treated and for collecting solution which flows off the treated limb for disposal. The first apparatus comprises a fluid containing tank, coupled to a fluid recirculation loop containing a pumping means and a differential pressure injector. An oxygen source is linked to the differential pressure injector via an ozone generator. Ozone gas is produced within the generator and entrained into the circulating fluid via the differential pressure injector to produce the high concentration aqueous ozone fluid.

US-A-2004/0016706 describes a method for producing aqueous ozone using a de-ionisation apparatus and an ozone generator. The system presents a fluid loop containing a Venturi injector. The fluid produced is intended to be applied to wounds.

These types of apparatus use potable water, the quality of which can vary depending on its source and method of processing. Water quality and chemical content can affect the concentration of aqueous ozone solution that can be achieved. The presence of oxidisable substances breaks down ozone entrained into the fluid until the substances have been fully oxidised. Purification of the potable tap water prior to ozonation decreases the amount of ozone and time period required to produce a specified ozone concentration.

Many methods exist for purifying water, such as reverse osmosis, distillation, filtration, electro-de-ionisation and ion-exchange. The apparatus is preferably a portable system and, as such, requires a method of purifying the water that is small, reliable, quick, relatively inexpensive and easy to use. The apparatus generally uses a source of oxygen to supply the ozone generator. This source can be from an oxygen concentrator, fixed supply line or oxygen cylinder. To allow the apparatus to be portable, a small, fixed volume, consumable aerosol is preferable, as is the de-pressurisation of the canister post use for disposal purposes.

A number of other solutions are know in the prior art for purifying water. For example U.S. Pat. No. B-5,024,766 describes a method for creating purified water involving the use of a de-ionisation system and an ozonation system. U.S. Pat. No. B-4,610,790 describes a method for de-ionising water, combined with a flush process prior to the start of each fluid production run. US-A-2003/0099584 describes a method for producing aqueous ozone using a de-ionisation apparatus and an ozone generator. It describes a method of de-ionising tap water prior to passing it through an ozone generator. U.S. Pat. No. B-6,080,313 describes a method for purging new de-ionisation cartridges with water to drain. The intent of the flush is to remove gross particulate that may have contaminated the ion-exchange media during transport or production.

Once de-ionisation is complete, if a canister has been used, it remains full of water. On removal, this fluid can spill onto the floor or the operator, creating a risk hazard. To overcome this type of problem ZA-A-988786 describes a method for removing residual water from a container which contains an ion exchange resin.

EP-A-0055590 discloses a mixed bed deioniser providing in-situ regeneration of cation and anion resins in a tank. EP-A-1393806 discloses a method for charging two stratified ion exchange resin beds into a condensate demineraliser. U.S. Pat. No. 2,902,155 discloses a method of water softening employing cation exchange materials.

The present invention relates to improvements in the usability of this type of apparatus and, in particular, the apparatus described in WO-A-2004/103452.

According to the invention there is provided a method of producing process fluid for disinfecting a surface, comprising the steps of:

flushing an ion exchange means with fluid from a fluid supply and directing the flushing fluid to waste;

de-ionising fluid from the fluid supply by passing it through the ion exchange means and directing the process fluid to storage means; and

characterised by the steps of ozonating the de-ionised fluid using an ozone generator provided with an oxygen source; and

purging the ion exchange means with gas to remove residual process fluid after the de-ionisation process has been completed.

The method preferably further comprises the step of purging fluid conduits connected to the ion exchange means.

The de-ionised fluid is preferably ozonated using an ozone generator provided with an oxygen source, and the oxygen source is preferably used to purge fluid from the ion exchange means.

The invention further provides apparatus for producing process fluid for disinfecting a surface, comprising:

an ion exchange means;

means for supplying fluid to the ion exchange means to flush the ion exchange means;

means for directing the flushing fluid to waste;

means for directing fluid to be de-ionised in the ion exchange means and passing the process fluid to storage means; and

characterised by an ozone generator provided with an oxygen source for ozonating the de-ionised fluid; and

means for directing gas to the ion exchange means to remove residual process fluid after the de-ionisation process has been completed.

The gas is preferably directed by the means for directing gas to remove residual process fluid from fluid conduits connected to the ion exchange means.

An oxygen source may be connected to supply oxygen to an ozone generator, said ozone generator being fluidly connected to a differential pressure injector.

Preferably means are provided to supply gas from the oxygen source to the ion exchange means to purge fluid from the ion exchange means.

The apparatus preferably further comprises a de-ionisation loop, said de-ionisation loop comprising the ion exchange means, the storage means, a pump and valve means, all of which are fluidly connected to enable fluid to be circulated around the de-ionisation loop.

A conductivity sensor may be provided for controlling the de-ionisation loop and may be located in the storage means.

The apparatus preferably further comprises an ozonation loop, said ozonation loop comprising the storage means, differential pressure means connected to the ozone generator, said pump and valve means, all of which are fluidly connected to enable de-ionised fluid to be circulated around the ozonation loop.

A timer means may be provided for controlling the ozonation loop.

Preferably the ion-exchange means is a consumable cartridge suitable for a single use.

The ion-exchange means is preferably connected to the apparatus by means of quick connect couplings.

The oxygen source is preferably a consumable cartridge suitable for a single use.

The present invention describes the use of a water purification means based on an ion-exchange mechanism to purify the water. The purification of the water allows a consistent level of high concentration aqueous ozone to be achieved from varying quality potable water supplies. The ion-exchange system is located in a dual loop, fluid recirculation arrangement with a differential pressure (Venturi) injector allowing for both water purification and aqueous ozone generation to be facilitated by a single pumping means.

Further, the fluid output line from the ion exchange/de-ionisation cartridge is connected to a purge line as well as connecting into the fluid re-circulation loop. The purge line is connected to waste, allowing organic compounds leaked from the ion-exchange media during storage to be washed to waste rather than into the process tank. Organic compounds within the process tank retard the ozonation process and hence the aqueous ozone concentration that can be achieved. Still further, a purge line is, at one end, connected to the upstream fluid supply loop of the ion exchange cartridge and connected to the system oxygen supply line at the other. The oxygen line provides oxygen to the ozone generator and subsequently the Venturi injector to produce the aqueous ozone solution. At the end of the ozonation phase, oxygen is directed through the purge line to clear the ion-exchange cartridge of liquid. The liquid is directed through the output purge line to waste, allowing the ion exchange cartridge to be removed/changed without spilling fluid. If the oxygen source is a small fixed volume consumable canister, the purge process empties the waste oxygen, allowing the canister to be disposed of in a standard waste stream.

The invention will now be described, by way of example only, with reference to and as shown in the accompanying drawings, in which:

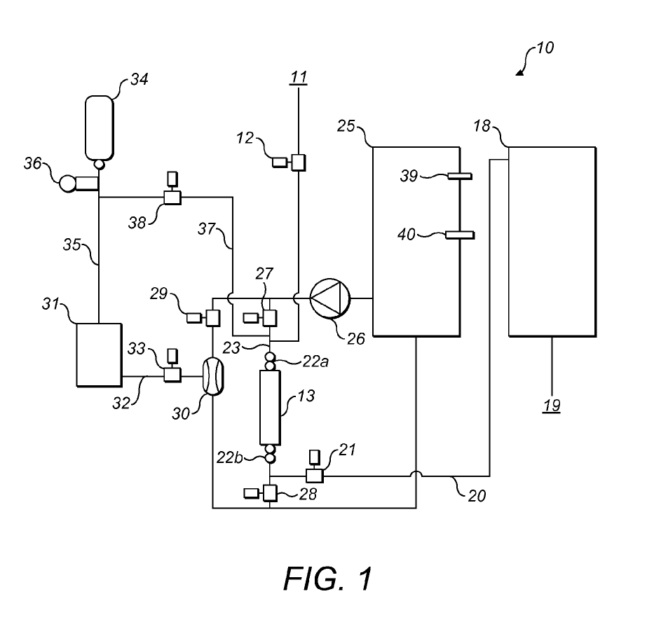

FIG. 1 is a schematic of the apparatus of the invention; and

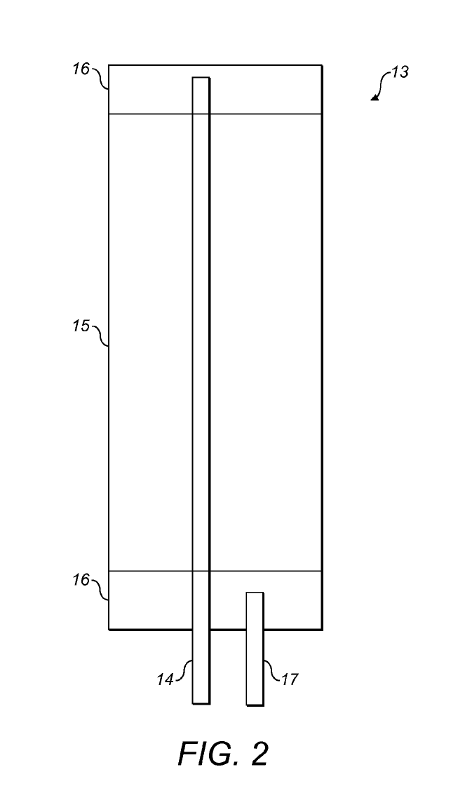

FIG. 2 is a schematic of the flow path through the de-ionisation cartridge to facilitate purging.

FIG. 1 is a schematic showing the elements of one embodiment of the apparatus which can be used in conjunction with the apparatus described in WO-A-2004/103452.

A supply of fluid, which is preferably water of potable quality, is provided to the apparatus 10 via fluid conduit 11. A valve 12 is located in fluid conduit 11. The fluid conduit 11 is fluidly connected to a de-ionisation cartridge 13, for example by means of a connection tee 23 and a quick connect coupling 22a at an inlet end of the cartridge 13.

FIG. 2 is a schematic representation of the internal structure of one suitable embodiment of the de-ionisation cartridge 13. Fluid enters the cartridge 13 via an inlet tube 14. De-ionisation media 15 is retained between two fluid permeable structures 16. Fluid moves through inlet tube 14, through media 15 and out of the cartridge via outlet tube 17.

The cartridge 13 is preferably connected to a waste tank 18, which has an outlet 19, by means of a flush line 20. A valve 21 is located in the flush line 20 and an outlet end of the cartridge is preferably connected to the flush line 20 by means of a further quick connect coupling 22b.

The apparatus comprises a de-ionisation re-circulation loop comprising a tank 25, a pump 26, a valve 27 at the inlet end of the cartridge 13, the cartridge 13 and a valve 28 at the outlet end of the cartridge 13, all of which are fluidly connected in series by means of conduits to enable the fluid to be circulated around the loop.

The apparatus further comprises an ozonation re-circulation loop comprising the tank 25, the pump 26, a valve 29 and a differential pressure injector 30, all of which are fluidly connected in series to form a loop by means of conduits, to enable the fluid to be circulated around the loop.

An ozone generator 31 is fluidly connected to the differential pressure injector 30 by means of a conduit 32, with a valve 33 located in the conduit 32. An oxygen canister 34 is fluidly connected to the ozone generator 31 by means of a conduit 35, with a pressure control valve 36 located in the conduit 35. A further conduit 37 connects conduit 35 to the de-ionisation re-circulation loop between valve 27 and the cartridge 13 and a further valve 38 is located in this conduit 37.

The tank 25 is provided with a level switch 39 and a conductivity sensor 40 of a suitable design.

The method of producing aqueous ozone solution by means of this apparatus is as follows. In a first step, the de-ionisation cartridge 13 is flushed through with fresh fluid. To effect this valve 12 is opened, and fresh fluid flows from the supply, along fluid conduit 11.

Valves 27, 38 and 28 remain closed and valve 21 is opened. The flushing fluid flows through the de-ionisation cartridge 13, via the connection tee 23 and the quick connect couplings 22a, 22b and into flush line 20 via valve 21. The flushing fluid enters waste tank 18, where it is directed to waste via outlet 19.

The de-ionisation cartridge 13 contains cationic and anionic de-ionisation resin which can leach organic molecules during storage. Flushing the cartridge 13 ensures that these leached molecules are flushed to waste. Without the flush process, leached organics can be taken into the process fluid to be reacted with gaseous ozone. The presence of organics retards the concentration of aqueous ozone that can be generated within a specified time period, as the ozone must first oxidise the organic molecules present. The de-ionisation cartridge flush phase continues for a period of time, typically related the bed volume of the cartridge 13. A timer is started when valve 21 opens and, on completion of the pre-determined flush time, valve 21 is closed.

The next stage is the de-ionisation of the fluid. Valve 28 is opened and fluid moves through cartridge 13, valve 28 and into tank 25. Water flows into tank 25 until the level switch 39 is activated. Valve 12 is closed and conductivity sensor 40 begins monitoring the conductivity of the fluid. Pump 26 is turned on and valve 27 is opened. In this stage, fluid is directed around the de-ionisation re-circulation loop from the tank 25, through the pump 26, the valve 27, the cartridge 13 and the valve 28 and back to the tank 25.

Fluid is re-circulated until it reaches a pre-determined conductivity set point, as measured by conductivity sensor 40. Pump 26 then turns off, and valves 27, 28 are closed.

The third stage of the process is the ozonation of the de-ionised fluid. To effect this, valve 29 is opened, pump 26 is turned on and fluid moves around the ozonation re-circulation loop from the tank 25, through the pump 26, the valve 29 and the differential pressure injector 30 and back to the tank 25. The flow of fluid through the differential pressure injector 30 creates a vacuum at the injector's gas inlet.

Valve 33 is opened, the ozone generator 31 is turned on and oxygen flows from oxygen canister 35, through pressure control valve 36 to the ozone generator 31. The oxygen gas passes through the ozone generator 31, where a proportion is reacted to produce ozone gas. The ozone gas is directed through valve 33 and is entrained into the recirculating fluid via differential injector 30. As the ozone generator 31 turns on, a timer is started. After a pre-determined length of time, valve 33 is closed, the ozone generator 31 is turned off, the pump 26 is stopped and valve 29 is closed. The resulting aqueous ozone solution is stored in tank 25 to be used in a surface decontamination process.

In the final stage of the process the de-ionisation cartridge 13 is purged to remove any remaining fluid. To effect this, valves 21, 38 are opened, a timer is started and oxygen from the canister 34 flows through de-ionisation cartridge 13, through valve 21 and into the waste tank 18. During purging, oxygen enters inlet tube 14 of the de-ionisation cartridge 13 and displaces water in the media 15 before passing back out of the cartridge 13 via outlet tube 17. Residual fluid from the de-ionisation process within cartridge 13 is thus displaced by the oxygen and is forced to waste tank 18. After a pre-determined time valves 21 and 38 are closed. The length of the timer is dependent on the volume of the oxygen canister 34 and size of cartridge 13. In the preferred embodiment, the oxygen canister 34 is a small, fixed volume aerosol. The timer is set to allow the aerosol to empty of oxygen via the de-ionisation cartridge 13, thus allowing the oxygen aerosol to be disposed of in a standard waste stream. A larger oxygen cylinder may be used, wherein the timer will be set to allow the de-ionisation cartridge 13 to be purged of water. Purging the de-ionisation cartridge 13 allows it to be removed without fluid spillage. In the preferred embodiment the de-ionisation cartridge 13 is a small, fixed volume consumable item, which once emptied of fluid, can be disposed of in a normal waste stream.

1-16. (canceled)

17. A method of producing process fluid for disinfecting a surface, comprising the steps of: flushing an ion exchange means with fluid from a fluid supply and directing the flushing fluid to waste;
de-ionising fluid from the fluid supply by passing it through the ion exchange means and directing the process fluid to storage means; and
characterised by the steps of ozonating the de-ionised fluid using an ozone generator provided with an oxygen source; and
purging the ion exchange means with gas to remove residual process fluid after the de-ionisation process has been completed.

18. A method as claimed in claim 17, further comprising the step of purging fluid conduits connected to the ion exchange means.

19. A method as claimed in claim 17, in which the oxygen source is used to purge fluid from the ion exchange means.

20. Apparatus for producing process fluid for disinfecting a surface, comprising:
an ion exchange means;
means for supplying fluid to the ion exchange means to flush the ion exchange means;
means for directing the flushing fluid to waste;
means for directing fluid to be de-ionised in the ion exchange means and passing the process fluid to storage means; and
characterised by an ozone generator provided with an oxygen source for ozonating the de-ionised fluid; and
means for directing gas to the ion exchange means to remove residual process fluid after the de-ionisation process has been completed.

21. Apparatus as claimed in claim 20, in which the gas is directed by the means for directing gas to remove residual process fluid from fluid conduits connected to the ion exchange means.

22. Apparatus as claimed in claim 20, further comprising an oxygen source connected to supply oxygen to an ozone generator, said ozone generator being fluidly connected to a differential pressure injector.

23. Apparatus as claimed in claim 22, further comprising means to supply gas from the oxygen source to the ion exchange means to purge fluid from the ion exchange means.

24. Apparatus as claimed in claim 20, further comprising a de-ionisation loop, said de-ionisation loop comprising the ion exchange means, the storage means, a pump and valve means, all of which are fluidly connected to enable fluid to be circulated around the de-ionisation loop.

25. Apparatus as claimed in claim 24, further comprising a conductivity sensor for controlling the de-ionisation loop.

26. Apparatus as claimed in claim 25, in which the conductivity sensor is located in the storage means.

27. Apparatus as claimed in claim 20, further comprising an ozonation loop, said ozonation loop comprising the storage means, differential pressure means connected to the ozone generator, said pump and valve means, all of which are fluidly connected to enable de-ionised fluid to be circulated around the ozonation loop.

28. Apparatus as claimed in claim 27, further comprising a timer means for controlling the ozonation loop.

29. Apparatus as claimed in claim 20, in which the ion-exchange means is a consumable cartridge suitable for a single use.

30. Apparatus as claimed in claim 29, in which the ion-exchange means is connected to the apparatus by means of quick connect couplings.

31. Apparatus as claimed in claim 20, in which the oxygen source is a consumable cartridge suitable for a single use.
